(19) United States
(12) Patent Application Publication
(10) Pub. No.: US 2004/0082665 A1
(43) Pub. Date: Apr. 29, 2004

(54) Method for treating stress or tension

(76) Inventor: Outi Maki-Ikola, Turku (FI)

Correspondence Address: FINNEGAN, HENDERSON, FARABOW, GARRETT & DUNNER, LLP, 1300 I STREET, NW, WASHINGTON, DC, 20005, US

(21) Appl. No.: 10/466,716

(22) PCT Filed: Jan. 22, 2002
(86) PCT No.: PCT/FI02/00047

(30) Foreign Application Priority Data
Jan. 22, 2001 (US) 09765329

(51) Int. Cl. A61K 31/137

(52) U.S. Cl. 514/650

(57) ABSTRACT

A method of treating stress and/or tension in a mammal by administering to the mammal an effective amount of 1,7,7-trimethyl-bicyclo[2.2.1]heptane derivative of Formula I

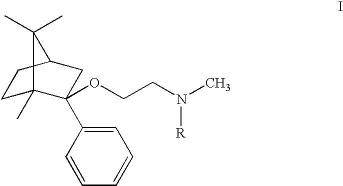

wherein R is hydrogen or methyl, or a pharmaceutically acceptable salt thereof.

### FIELD OF THE INVENTION

The present invention relates in general to a method for treating stress and/or tension in a mammal. More particularly, the invention relates to a method for treating stress and/or tension in a mammal by administering to the mammal an effective amount of 1,7,7-trimethylbicyclo[2.2.1]heptane derivative of Formula I

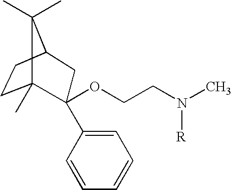

wherein R is hydrogen or methyl, or a pharmaceutically acceptable salt thereof.

Additional objects and advantages of the invention will be set forth in part in the description, which follows, and in part will be obvious from the description, or may be learned by practice of the invention. The objects and advantages of the invention will be realised and attained by means of the elements and combinations particularly pointed out in the appended claims.

### BACKGROUND OF THE INVENTION

The active ingredients of this invention, (1R,2S,4R)-(−)- 2-phenyl 2-(dimethylaminoethoxy)-1,7,7-trimethyl-bicyclo[2.2.1]heptane, known as deramciclane, and (1R,2S,4R)-(−)-2-phenyl-2-(methylaminoethoxy)-1,7,7-trimethyl-bicyclo[2.2.1]heptane, known as N-desmethylderamciclane, and their pharmaceutically acceptable acid addition salts with inorganic and organic acids generally used for the purpose, fall within the disclosures of U.S. Pat. No. 4,342,762 and International Patent Application No. WO 98/17230, respectively, which are both incorporated herein by reference.

These compounds are selective serotonin 5HT2A- and/or 5HT2C-receptor antagonists. They have shown anxiolytic-like effects in animal test models.

### DESCRIPTION OF THE INVENTION

Applicants have surprisingly discovered that the compounds of Formula (I) have a therapeutic effect on the ability to tolerate stress and tension in a mammal. Accordingly, an object of the present invention is a method for treating stress and/or tension in a mammal by administering to the mammal an effective amount of a compound of Formula (I) or a pharmaceutically acceptable salt thereof.

According to the present invention stress is either an acute or chronic condition associated with a variety of types of symptoms, such as anxiety, nervousness, inner tension, insomnia, concentration difficulties, memory impairment, muscle tension and palpitation.

According to the present invention tension is either an acute or chronic condition associated with either muscular tension (associated with symptoms like twitchings, stiffness, myoclonic jerks, grinding of teeth, unsteady voice or increased muscular tone) or other tension (associated with symptoms like feelings of tensions, fatigability, startle response, moving to tears easily, trembling, feelings of restlessness or inability to relax) or both.

Stress and tension may also be associated with an affective disorder, such as depression or with an anxiety disorder, such as Generalized Anxiety Disorder (GAD), Social Anxiety Disorder (SAD), Panic Disorder (PD), Obsessive Compulsive Disorder (OCD), Post-Traumatic Stress Disorder (PTSD) or agoraphobia.

For the purposes of this disclosure and claims the term “treatment” means treatment in order to cure or alleviate the disease or its symptoms, and to treatment in order to prevent the development or the exacerbation of the disease or its symptoms.

Pharmaceutically acceptable salts of the compound of Formula (I) can be formed with inorganic acids, e.g. hydrohalogenic acid such as hydrochloric acid or hydrobromic acid, sulfuric acid, phosphoric acid or nitric acid, or organic acids e.g., tartaric acid, succinic acid, malic acid, maleic acid, fumaric acid, citric acid, or lactic acid. Salt with fumaric acid is preferred.

Pharmaceutical compositions containing a compound of Formula (I) or a pharmaceutically acceptable salt thereof as the active ingredient include the usual oral dosage forms, such as tablets, capsules, and liquid preparations. In oral dosage forms, the active ingredient can be mixed with suitable pharmaceutically acceptable excipients, such as starch, lactose, sucrose and magnesium stearate, in accordance with conventional pharmaceutical practice.

The precise amount of the drug to be administered to a mammal for the treatment of stress is dependent on numerous factors known to one skilled in the art, such as the compound to be administered, the general condition of the patient, the condition to be treated etc. For example, the usual recommended oral daily dose of deramciclane would be about 5-150 mg/day, or about 10-60 mg/day, or about 30-60 mg/day, or about 30 mg/day.

The invention will be further clarified by the following example, which is intended to be purely exemplary of the invention.

### EXAMPLE 1

The effects of deramciclane were studied in a randomised placebo-controlled double-blind study. The subjects were randomly assigned to four parallel groups to receive one tablet twice daily (b.i.d) of a placebo, 5 mg (=10 mg/day), 15 mg (=30 mg/day), or 30 mg (=60 mg/day) deramciclane.

The efficacy of deramciclane on the ability to tolerate stress was analysed by the Visual Analogue Scale (VAS).

Administration of deramciclane improved the ability to tolerate stress when measured by the VAS. The ability to tolerate stress was 29%, 54% and 43% better in patients receiving deramciclane 5 mg b.i.d, 15 mg b.i.d, and 30 mg b.i.d., respectively, than in patients receiving placebo.

### EXAMPLE 2

The efficacy and safety of deramciclane was studied in the treatment of tension as compared to placebo. A randomised plasebo-controlled double-blind parallel group 8-week study design was used.

Patients were asked if they had muscular tension (twitchings, stiffness, myoclonic jerks, grinding of teeth, unsteady voice or increased muscular tone) or other tension (feelings of tension, fatigability, startle response, moving to tears easily, trembling, feelings of restlessness or inability to relax). This tension was related to be not present, mild, moderate, severe or very severe.

In addition, patients' tension was also rated with the tension scale 1-6:

0—placid

2—occasional feelings of edginess and ill defined discomfort

4—continuous feelings of inner tension

6—unrelated dread or anguish

Moderate/severe or very severe muscular tension was present in patients at baseline in 82% placebo), 61% (15 mg deramciclane bid) and 68% (30 g deramciclane bid) of patients; the corresponding figures after 8 weeks of treatment were 38%, 18% and 15%. Thus deramciclane improved muscular tension in patients.

Moderate/severe or very severe other tension was present in patients at baseline in all patients. After 8 weeks of treatment this was present in 43% (placebo), 25% (15 mg bid deramciclane) and 24% (30 mg bid deramciclane) patients. Thus, deramciclane improved also this kind of tension in patients.

48%, 35% and 47% of patients in the placebo, 15 mg bid deramciclane and 30 mg bid deramciclane groups had tension scale number at least 4 at baseline. After 8 weeks of treatment the corresponding numbers were 12%, 5% and 11%.

Although the invention has been illustrated by the preceding example, it is not to be construed as being limited to the materials employed therein; rather, the invention is directed to the generic area as herein disclosed. Various modifications and embodiments thereof can be made without departing from the spirit or scope thereof.

We claim:

1. A method of treating stress and/or tension in a mammal, comprising administering to said mammal an effective amount of at least one compound of Formula (I) 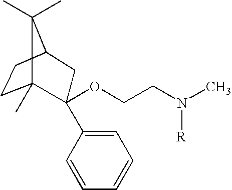
wherein R is hydrogen or methyl, or a pharmaceutically acceptable salt of the compound of Formula (I).

2. The method of claim 1, wherein the stress or tension is associated with an affective disorder.

3. The method of claim 2, wherein the affective disorder is depression.

4. The method of claim 1, wherein the stress or tension is associated with an anxiety disorder.

5. The method of claim 4, wherein the anxiety disorder is Generalized Anxiety Disorder.

6. The method of claim 4, wherein the anxiety disorder is Social Anxiety Disorder.

7. The method of claim 4, wherein the anxiety disorder is Panic Disorder.

8. The method of claim 4, wherein the anxiety disorder is agoraphobia.

9. The method of claim 4, wherein the anxiety disorder is Obsessive Compulsive Disorder.

10. The method of claim 6, wherein the anxiety disorder is Post-Traumatic Stress Disorder.

11. The method of claim 1, wherein the mammal is human.

12. The method of claim 1, wherein at least one compound is deramciclane or a pharmaceutically acceptable salt thereof.

13. The method of claim 1, wherein about 5 to about 150 mg/day of at least one compound claimed in claim 1 is administered.

14. The method of claim 13, wherein the amount administered is about 10 to about 60 mg/day.

15. The method of claim 14, wherein the amount administered is about 30 mg/day.
